(19) United States
(12) Patent Application Publication
(10) Pub. No.: US 2026/0000550 A1
(43) Pub. Date: Jan. 1, 2026

(54) COMPRESSION SLEEVE WITH REMOVEABLE ABSORBENT MATERIAL

(71) Applicants: Rita BROWN-HOLLOWAY, Orange, CT (US); Tyrone HOLLOWAY, Orange, CT (US)

(72) Inventors: Rita BROWN-HOLLOWAY, Orange, CT (US); Tyrone HOLLOWAY, Orange, CT (US)

(21) Appl. No.: 18/754,446

(22) Filed: Jun. 26, 2024

(51) Int. Cl. A61F 13/10; A41D 13/00

(52) U.S. Cl. CPC A61F 13/10; A41D 13/0002; A41D 27/10; A63B 71/12

(57) ABSTRACT

An article adapted to be worn by an athlete comprising a combination compression sleeve and absorbent material, where the absorbent material is detachably connected to the compression sleeve. The compression sleeve includes an anti-slip structure that includes an end with an arm hole, such that, a user may slip their arm though the arm hole of the anti-slip structure and then through the compression sleeve. The anti-slip structure extends over the user's shoulder to prevent the compression sleeve from slipping downward on the user's arm, which could occur otherwise due to wiping actions (e.g., wiping sweat from head, face, neck, and body) down the absorbent material exerting downward pressure on the compression sleeve.

### BACKGROUND

### 1. Field of the Invention

The present disclosure is related to compression sleeves. More particularly, the present disclosure is related to a compression sleeve that includes an absorbent material that is removably attachable to an inside portion of the compression sleeve.

### 2. Description of Related Art

Sweating profusely during a sporting event, during workouts, and other activities is a common problem for athletes as the sweat, while beneficial in cooling down the body, can become a nuisance during the activity. For example, during a game, if the athlete is sweating profusely, the sweat can fall on the floor and cause a slipping hazard or the sweat can get in the athlete's eyes obscuring their vision or could simply be a distraction to the athlete.

As a result, many athletes wear absorbent headbands and wristbands to conveniently manage sweat during activity and game play. A headband, however, will only passively absorb sweat from the narrow, targeted area of the forehead and as such, are ineffective at absorbing sweat from other critical areas of the body such as the face, neck, and body. To manage other areas of the body, athletes have resorted to wearing wristbands to absorb sweat on other areas of the body. However, wristbands are narrow and short (e.g., 6-inches in length), and as such, are limited in their capacity to wipe away and collect significant amounts of sweat from the body. For athletes at the collegiate or professional level, these are often marginally effective and need to be replaced numerous times per game.

Additionally, with the increased usage of compression sleeves, the use of wristbands is very difficult for athletes. Some athletes believe the mild compression provided by a compression sleeve helps to keep the athletes shooting arm warm and improves circulation. Additionally, some studies have shown improved circulation and reduced soreness in the limb on which the compression sleeve is worn. In any event, the use of compression sleeves is increasing for athletes at the collegiate or professional level. However, compression sleeves are typically made from a combination of spandex and nylon to form a lightweight cover that can stretch and is very smooth or slippery to the touch. When an athlete places a wristband (typically made from an absorbent cloth material), the intense movement and interaction with other athletes on the court can cause these wristbands to fall off due to a very low coefficient of friction between the wristband and the compression sleeve. Unfortunately, the compression sleeve itself is unable to absorb significant amounts of sweat from the body, which was never a function compression sleeves were designed to serve.

One configuration U.S. Pat. No. 9,936,751 (the '751 patent) provides a sweat absorption material that is designed to essentially cover an athlete's entire forearm. While this configuration does allow for greater sweat absorption than a wristband only, the arm sleeve is not a compression sleeve and will therefore not provide the benefits of compression sleeves as previously discussed. In another embodiment, the '751 patent describes that a compression sleeve can be worn under the arm sleeve, which is designed to fit over top of the compression sleeve. However, this becomes a very bulky and movement restrictive garment, which is not advantageous for athletes at the collegiate and professional levels. It would also be counterproductive to the whole concept of sweat reduction, as it might make the arm(s) much hotter resulting in additional sweating during exercise. Individuals playing basketball, for example, would refuse to use such a product as it would impede their ability to move their arm(s) freely as with a compression sleeve only. Additionally, fitting the arm sleeve over top of the compression sleeve will not provide a firm hold for the arm sleeve especially during highly competitive play where the arm sleeve would move over the slippery surface of the compression sleeve. Any sliding of the arm sleeve over the surface of the compression sleeve would be unacceptable and distracting for the athlete wearing the combination of sleeves.

As a result of the above-listed shortcomings, athletes, more often, than not, simply resort to using towels to manage profuse sweating during game play and sports activity. However, this requires them to interrupt their respective sports activities to go to their bags or benches to retrieve a towel to wipe away the sweat from their body. This, however, can interrupt game flow, sports zone, and success outcomes. For games, the move to the bench to perform this activity can only be done during a time out when the game flow is paused.

Accordingly, there is a need for a new type of absorbent structure that overcomes, alleviates, and/or mitigates one or more of the aforementioned and other deleterious effects of prior art sweat absorbing devices.

### SUMMARY

Accordingly, what is desired is a device that can be used to absorb liquid (sweat), matter, shock or force from or to different parts of the body and can be effectively used in conjunction with a compression sleeve.

It is further desired to provide a combination compression sleeve and sweat absorbing device that can essentially be used throughout a game without requiring the device to be replaced or changed during a game.

It is still further desired to provide a combination compression sleeve and sweat absorbing device where the sweat absorbing device is detachable from the compression sleeve.

It is also desired to provide a combination compression sleeve and sweat absorbing device that will not move relative to the arm or be displaced during game play and use.

In one configuration, a combination compression sleeve and absorbent material strip is provided that allows for the benefits of using a compression sleeve, but also allows for a significant amount of absorbent material to be affixed into the compression sleeve such that it would not need to be replaced during a competitive game. In one embodiment, the compression sleeve is provided with location on an inner arm side of the compression sleeve on which the absorbent material can be affixed. It is contemplated that the absorbent material can essentially run the length of the forearm on one side allowing the athlete to use the absorbent material to wipe away sweat from the athlete's head, face, neck and body and is of a sufficient length to absorb far more than smaller existing wrist- and head bands. In this embodiment, the absorbent material is essentially an open-faced strip of material removably affixed to the surface of the compression sleeve. It will be noted that the absorbent material may run a longitudinal length of the compression sleeve or may be formed as multiple inserts extending at least partially radially around the compression sleeve. There are many configurations that could be used for the absorbent material.

It is further contemplated that a channel can be provided, such as in the form of a track, into which the absorbent material can be slid. In another example, the absorbent material could be affixed to the compression sleeve via a hook and loop fastener configuration. Likewise, a combination of the two may be utilized. In any event, the absorbent material is firmly affixed to the compression sleeve such that the absorbent material cannot move relative to the compression sleeve. The tract provides an open top, which allows for a sweat-logged bar to be easily removed or popped in and out of the tract and easily replaced with a clean, dry replacement sweat bar. The absorbent material can be provided as a cloth material and is thick relative to the compression sleeve.

In another configuration, the compression sleeve itself can be held more firmly in place on the arm than existing compression sleeves due to the provision of double arm holes. This serves to hold the compression sleeve to the arm such that when it is used to wipe away sweat, the compression sleeve does not move or slip downward on the arm, even when pulled.

For this application the following terms and definitions shall apply:

The terms “first” and “second” are used to distinguish one element, set, data, object or thing from another, and are not used to designate relative position or arrangement in time.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The terms “coupled”, “coupled to”, “coupled with”, “connected”, “connected to”, and “connected with” as used herein each mean a relationship between or among two or more devices, apparatus, components, systems, subsystems, and/or means, constituting any one or more of (a) a connection, whether direct or through one or more other devices, apparatus, components, systems, subsystems, or means, (b) a communications relationship, whether direct or through one or more other devices, apparatus, components, systems, subsystems, or means, and/or (c) a functional relationship in which the operation of any one or more devices, apparatus, components, systems, subsystems, or means depends, in whole or in part, on the operation of any one or more others thereof.

In one configuration, an article for providing sweat absorption for a user and providing compression to a body limb is provided, the article comprising: a compression sleeve having a proximal end with an opening into which a user can insert a limb, and a distal end with an opening such that when the compression sleeve is fitted on the limb, the end of the limb protrudes from the distal end opening, and the an absorbent material form as a strip of material extending along a longitudinal length of the compression sleeve. The article is provided such that a width of the absorbent material is less than a diameter of the compression sleeve and a length of the absorbent material is less than a length of the compression sleeve. Finally, the article is provided such that the absorbent material is detachably connected to the compression sleeve.

In another configuration, an article for providing sweat absorption for a user and providing compression to a body limb is provided, the article comprising: a compression sleeve having a proximal end with an opening into which a user can insert a limb, and a distal end with an opening such that when the compression sleeve is fitted on the limb, the end of the limb protrudes from the distal end opening, and an absorbent material form as a strip of material extending along a longitudinal length of the compression sleeve, wherein a width of the absorbent material is less than a diameter of the compression sleeve and a length of the absorbent material is less than a length of the compression sleeve. The article further comprises an anti-slip structure extending from the proximal end of the compression sleeve, the anti-slip structure comprising a portion of the proximal end of the compression sleeve extending longitudinally past the proximal opening and formed as a longitudinally extending inner structure to form an upper end, and an opening formed in the upper end. The article is provided such that it is adapted to facilitate a user extending their arm through the opening formed in the upper end and then extending their arm through the proximal opening of the compression sleeve such that the upper end is adapted to sit over top of the user's shoulder when the compression sleeve is fully advanced over the arm and the upper end over the shoulder, the upper end preventing the compression sleeve from moving downward relative to the user's arm when in use.

The above-described and other features and advantages of the present disclosure will be appreciated and understood by those skilled in the art from the following detailed description, drawings, and appended claims.

### DESCRIPTION OF THE DRAWINGS

FIG. 1 is an illustration of the combination compression sleeve and absorbent material showing the outside edge (facing away from the body) of the article according to a configuration of the invention;

FIG. 2 is an illustration of the combination compression sleeve and absorbent material, showing the inside edge (facing toward the body) of the article, according to FIG. 1;

FIG. 3 is an illustration of the absorbent material disconnected from the compression sleeve according to a first configuration based on FIG. 2;

FIG. 4 is an illustration of the absorbent material disconnected from the compression sleeve according to a second configuration based on FIG. 2;

FIG. 5 is an illustration of the absorbent material connected to the compression sleeve according to FIG. 4;

FIG. 6 is a cross-sectional diagram along Section A-A according to FIG. 5;

FIG. 7 is an illustration of the absorbent material disconnected from the compression sleeve according to FIG. 3 and further includes an absorbent material extending around a wrist portion of the compression sleeve;

FIG. 8 is an illustration of the absorbent material with a back bar and additional shoulder structure according to the configuration of FIG. 1; and

FIG. 9 is an illustration of the absorbent material with an additional compression sleeve according to the configuration of FIG. 8.

### DETAILED DESCRIPTION

Referring now to the drawings, wherein like reference numerals designate corresponding structure throughout the views.

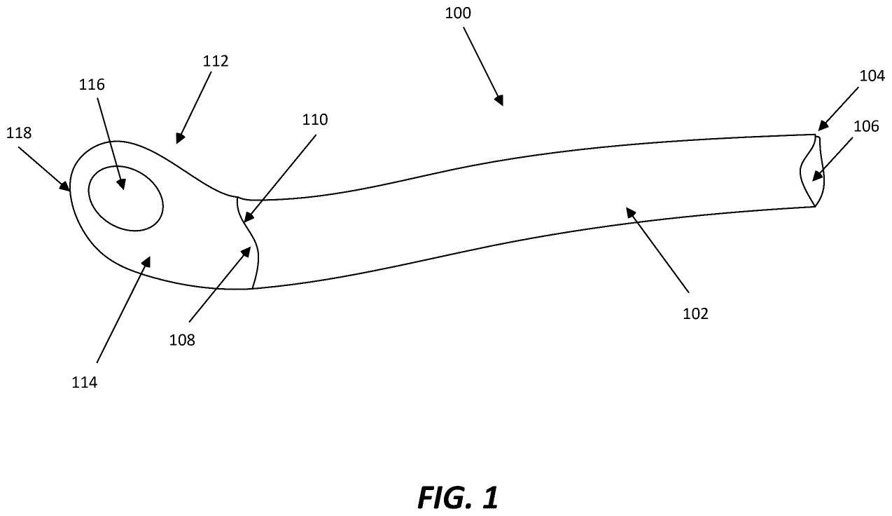

Referring to the drawings and in particular, to FIG. 1, an article 100 is provided that comprises a compression sleeve 102 including a distal end 104 with a distal opening 106, and a proximal end 108 with a proximal opening 110. It is contemplated that the compression sleeve may comprise a relatively thin, nylon and spandex material and applies an even compression to the limb within the sleeve.

The article 100 further comprises an anti-slip structure 112 that comprises an elongated material 114 extending from the proximal end 108. The elongated material further includes an opening 116 positioned therein to allow a user to insert their arm through opening 116.

In function, a user would pass their hand and arm through the opening 116 and then into proximal opening 110 and finally out through distal opening 106. When article 100 is fully seated on the arm, the upper end 118 elongated material 114 will sit over the top of the user's shoulder with the elongated material 114 running along an inner side of the user's arm. This anti-slip structure 112 can be made of the same material as the compression sleeve 102 and can be formed ergonomically to comfortably conform to the user's shoulder. In this manner the compression sleeve will be prevented from slipping or traveling down the user's arm as the upper end 118 prevents any downward longitudinal movement.

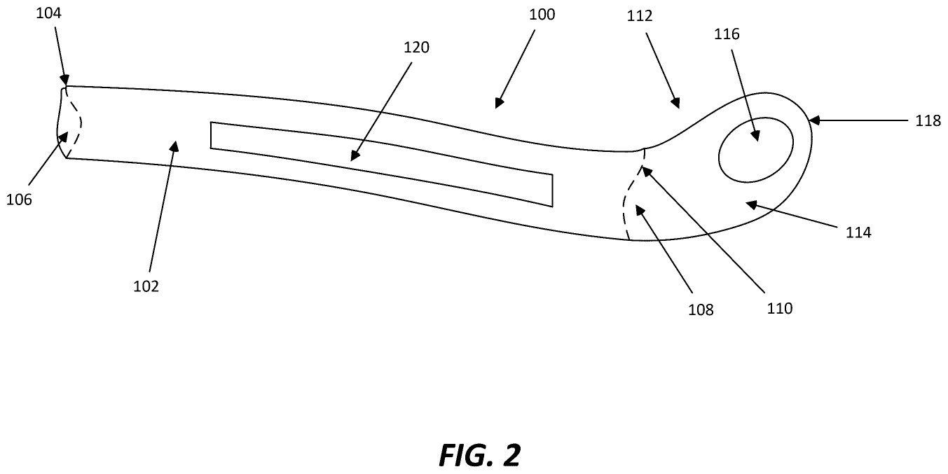

FIG. 2 is a view of article 100 looking at the inside portion (facing toward the body) of the compression sleeve 102 and showing the absorbent material 120. Based on the configuration, a user would put their arm through the opening 116 and then into the proximal opening 110 with the user's hand extending out of distal opening 106. In this configuration, the absorbent material 120 faces toward the user making it easy to use the absorbent material 120 to wipe away sweat from the user's head, face, neck and body. It is contemplated that the absorbent material 120 may comprise a fabric or cloth material.

The absorbent material 120 is situated or positioned along the inner seam of the compression sleeve 102, however, it is contemplated that the compression sleeve 102 could be rotated axially around the arm to radially position the absorbent material 120 on the arm as desired (e.g., the compression sleeve 102 could be rotated such that the absorbent material 120 is radially positioned towards the inner top of the arm) being limited only by the slip structure 112 and upper end 118 that loops over the user's shoulder. It should be noted that the absorbent material could run along a longitudinal length of the compression sleeve or may be formed as a absorbent material extending at least partially radially around the compression sleeve. Additionally, rather than only one absorbent material, the compression sleeve could be adapted to function with multiple discrete absorbent materials that are coupled to the compression sleeve. For example, a relatively short absorbent material could be provided as the inside of the wrist and a second longitudinally extending absorbent material could be provided on the inside edge of the forearm. While the figures illustrate one configuration, the invention is not limited to only a longitudinally extending absorbent material.

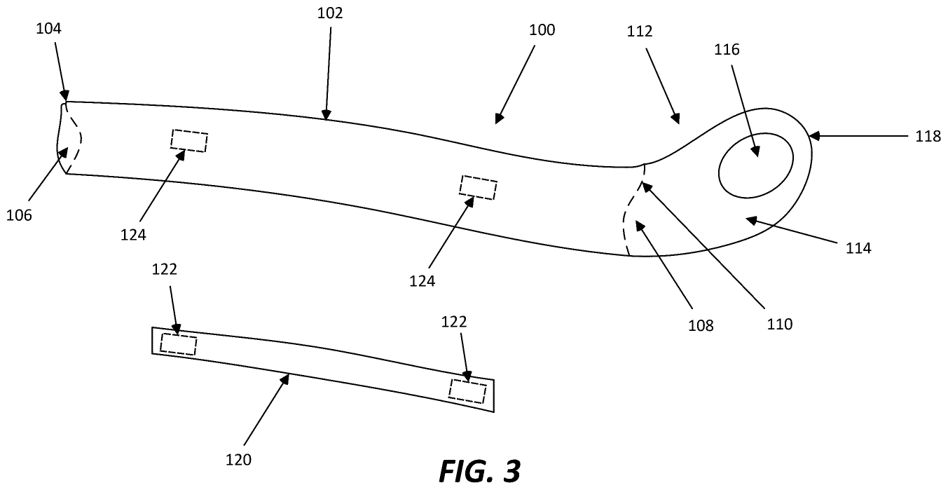

FIG. 3 illustrates a first embodiment of the invention according to the configurations of FIGS. 1 and 2. Article 100 is shown with the absorbent material 120 disconnected from the compression sleeve 102. Also illustrated is a securing mechanism that comprises a hook fastener 122 that is adapted to engage with a loop fastener 124. While only two sets of hook 122 and loop 124 fasteners are shown, it is contemplated that any number of fasteners may be positioned along a longitudinal length of the absorbent material 120. In another configuration, a hook 122 and loop 124 fastener may extend along a complete longitudinal length (or any portion thereof) of the absorbent material 120 to ensure it is firmly held to the compression sleeve 102.

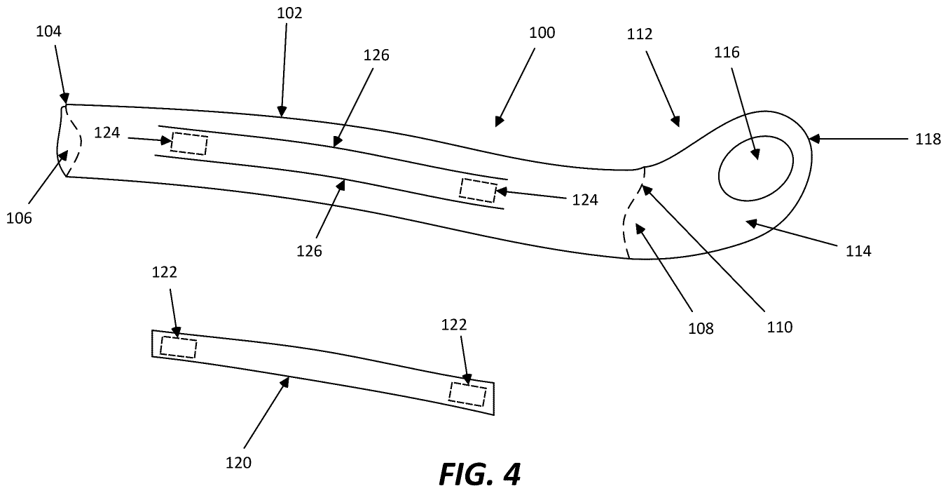

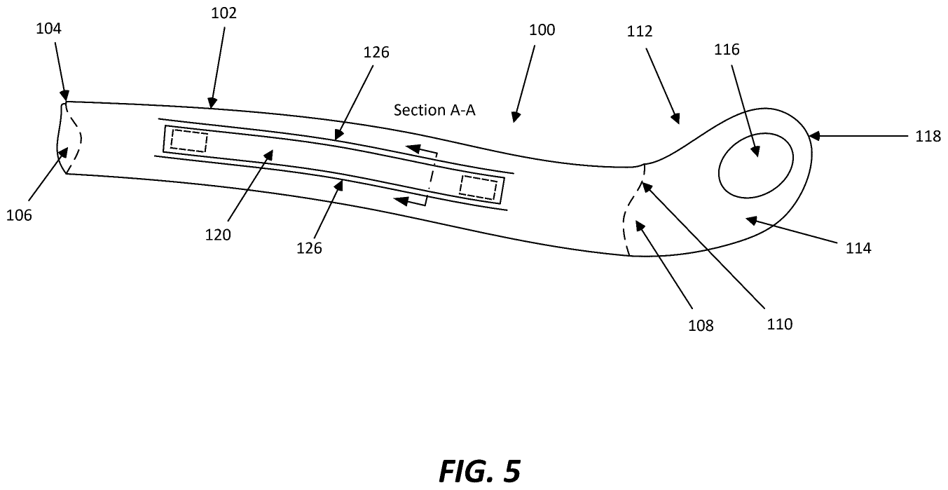

FIGS. 4 and 5 illustrate a second embodiment of the invention according to the configurations of FIGS. 1 and 2. Article 100 is again shown with the absorbent material 120 disconnected from the compression sleeve 102 (FIG. 4). In this configuration, track 126 is shown on the surface of compression sleeve 102 into which the absorbent material 120 is adapted to be positioned. The track 126 may be affixed (e.g., sewn) into the compression sleeve 102. In one configuration, track 126 may comprise a plastic material that is semi-rigid but allows for flexibility to conform to the curvature of the athlete's arm.

FIG. 5 shows the absorbent material 120 inserted into track 126 and held in place via a securing mechanism, which may, in one configuration, comprise a hook and loop fastener.

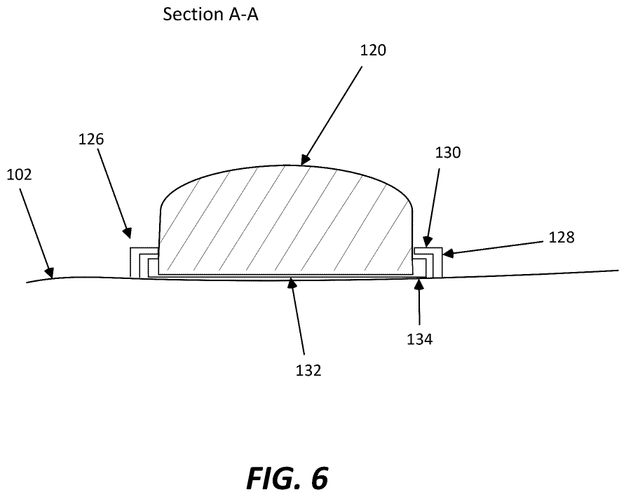

Referring now to FIG. 6, a cross-section of Section A-A (FIG. 5) is illustrated. The track 126 is shown comprising an upstanding wall 128 and formed with an undercut 130.

The absorbent material is further shown having a backing 132, that includes ends 134 that are adapted to engage with the undercut 130. The drawings are not shown to scale, and it is contemplated that the ends 134 may comprise a very low profile structure or tab (e.g., the backing may or may not extend upward around the side of the absorbent material 120). It is contemplated that the absorbent material 120 may be affixed to the backing by an adhesive.

Additionally, it is contemplated that the backing 132 may comprise a semi-rigid material that is deformable allowing the absorbent material 120 to easily be inserted and removed from the track.

In one configuration, backing 132 is provided with a securing mechanism as previously described. In other configuration, the backing 132 may be sized to fit tightly within track 126 such that the absorbent material 120 is held in place via a friction fit between the ends 134 and the undercut. To insert or remove the absorbent material 120, a user simply has to grab the absorbent material 120 at one end and pull upward and the backing 132 will deform allowing removal. Alternatively, the user could simply slide the absorbent material 120 out of an end of the track 126. In any event, a quick and easy change of the absorbent material 120 is possible in just a few seconds if the athlete needs to replace the absorbent material 120 during a game (e.g., during a time out).

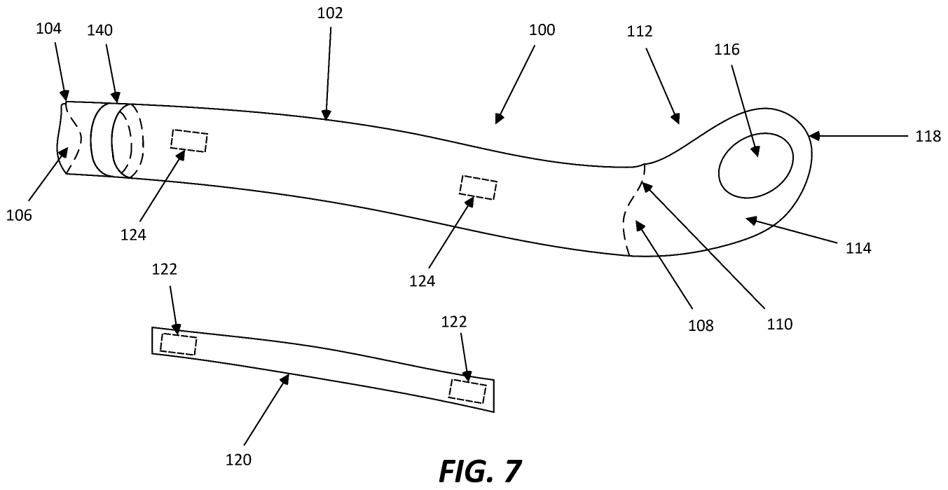

FIG. 7 is a configuration similar to FIG. 3 but further includes an absorbent material 140 positioned around a wrist portion of the compression sleeve 102. In this configuration, it is contemplated that the absorbent material 140 may be removable from the compression sleeve 102 and may further include a securing mechanism as previously discussed. In one configuration, the absorbent material 140 may extend around a radial portion of the compression sleeve 102 (e.g., 120 degrees, or 180 degrees). In this configuration, the securing mechanism may include, for example, the hook and loop fastener or the track or both. In another configuration, the absorbent material 140 may extend completely around compression sleeve 102. In this configuration, the absorbent material 140 may comprise a stretchable material that may be used with the securing mechanism. The absorbent material 140 can also serve to absorb the shock/force of a basketball rim exerted on an athlete's dorsal wrist during dunks or other types of physical contact.

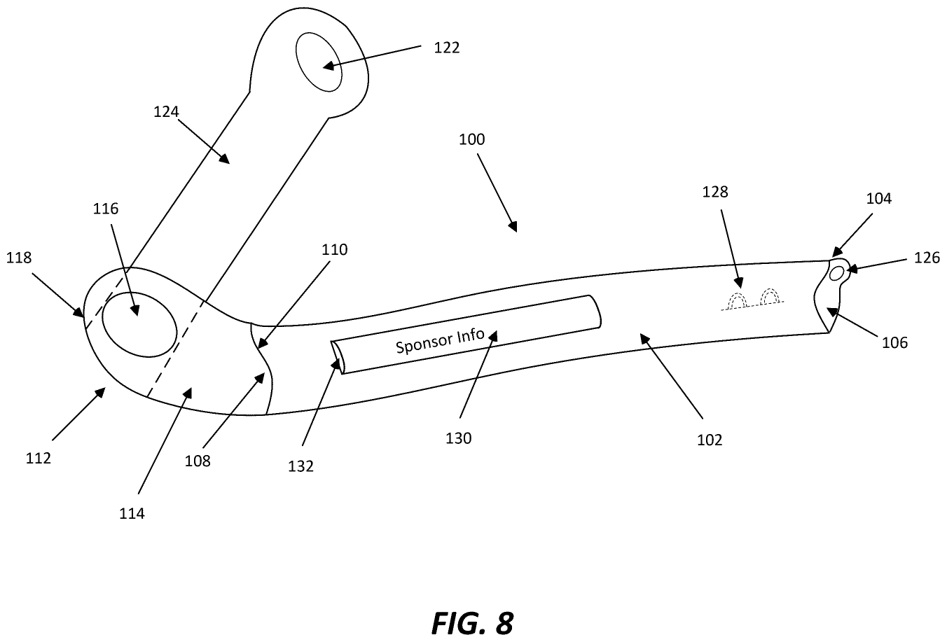

Referring now to FIG. 8, an alternate configuration to article 100 shown in FIG. 1 is provided. In this configuration, a back bar 124 is connected to upper end 118 and terminates with a shoulder structure 136 having an opening 122 positioned therein.

In function, a user would pass their left arm and hand through opening 122 so that it is fully seated over the top of the user's left shoulder; where opening 122 is also attached along its rear seam to a back bar 124 comprised of an elongated piece of material, (that could also be thick and absorbent). This configuration will function to more securely hold the compression sleeve in place preventing any type of downward movement.

In another configuration, the compression sleeve also features a thumb hole 126 for securing and/or stabilizing the compression sleeve on the arm during use.

In still another configuration, the compression sleeve also features 128 silicone or rubberized stress pulls or fidgets to help athletes release stress and anxiety during breaks or while seated on the bench.

In yet another configuration, the compression sleeve also features a clear slot 130 for inserting through an opening 132 various placards or pictures displaying athletes' NIL deals or other types of advertising.

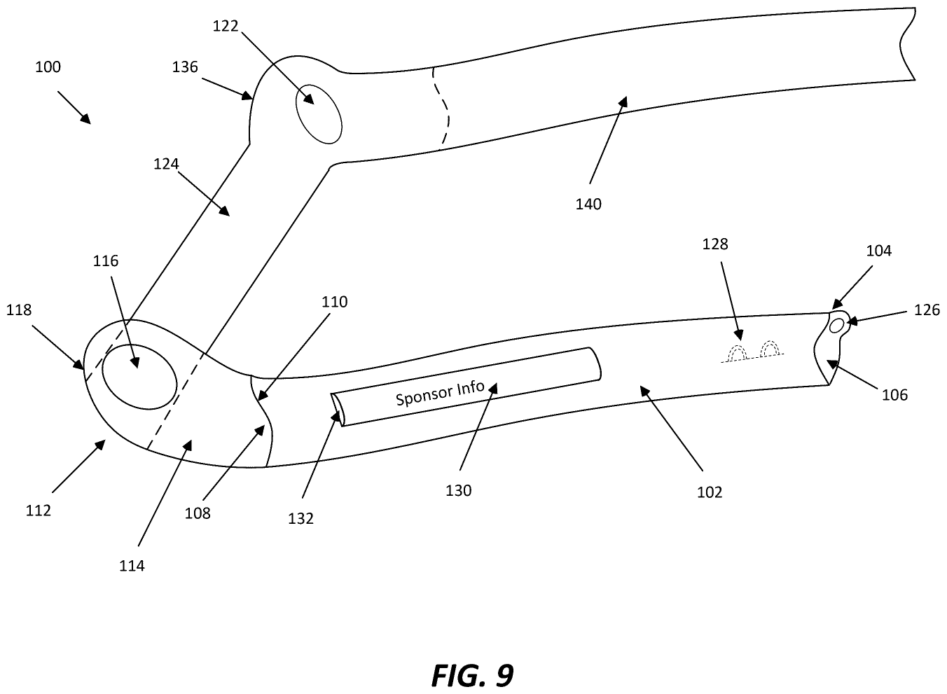

Turning now to FIG. 9 still another configuration is shown. In this embodiment, a second arm 140 is shown connected to the shoulder structure 136. The second arm 140 may include some or all the various features discussed in connection with the previous FIGURES and will not be redescribed here. Likewise, the back bar 124 may include a removable sweat insert as discussed in connection with the compression sleeves or may simply comprise the same material forming the compression sleeves.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. An article for providing sweat absorption for a user and providing compression to a body limb comprising:
a compression sleeve having a proximal end with an opening into which a user can insert a limb, and a distal end with an opening such that when the compression sleeve is fitted on the limb, the end of the limb protrudes from the distal end opening;
an absorbent material formed as a strip of material extending along a portion of the length of the compression sleeve;
wherein a width of the absorbent material is less than a diameter of the compression sleeve and a length of the absorbent material is less than a length of the compression sleeve;
wherein the absorbent material is detachably connected to the compression sleeve.

2. The article of claim 1, wherein the absorbent material is formed as an insert, the article further comprising:
a tract positioned on a surface of the compression sleeve;
wherein the insert is adapted to be held in the tract.

3. The article of claim 2, wherein the insert is provided with a backing material that is rigid or semi-rigid.

4. The article of claim 3, wherein the backing is formed of a flexible plastic material.

5. The article of claim 3, further comprising:
a securing mechanism positioned on a surface of the compression sleeve and within the track;
wherein, when the insert is fully inserted into the tract, the securing mechanism holds the insert in place in the tract.

6. The article of claim 5, wherein the track comprises a upstanding wall with an undercut, and the backing is formed with an edge that is adapted to interact with the undercut.

7. The article of claim 5, wherein the securing mechanism comprises a hook and loop fastener positioned on an outer surface of the compression sleeve and an inner surface of the insert.

8. The article of claim 1, further comprising a securing mechanism adapted to hold the absorbent material in a fixed position relative to the compression sleeve.

9. The article of claim 8, wherein the securing mechanism comprises a hook and loop fastener positioned on an outer surface of the compression sleeve and an inner surface of the absorbent material.

10. The article of claim 9, wherein the hook and loop fastener extends substantially the length of the absorbent material.

11. The article of claim 8, wherein the absorbent material is provided with a backing material that is rigid or semi-rigid.

12. The article of claim 1, further comprising:
an anti-slip structure extending from the proximal end of the compression sleeve, the anti-slip structure comprising:
a portion of the proximal end of the compression sleeve extending longitudinally past the proximal opening and formed as a shoulder structure to form an upper end; and
an opening formed in the upper end;
wherein the article is adapted to facilitate a user extending their arm through the opening formed in the upper end and then extending their arm through the proximal opening of the compression sleeve such that the upper end is adapted to sit over top of the user's shoulder when the compression sleeve is fully advanced over the arm and the upper end over the shoulder, the upper end preventing the compression sleeve from moving downward relative to the user's arm when in use.

13. The article of claim 12, wherein the shoulder structure comprises a first shoulder structure and the opening in the upper end of the first shoulder structure comprises a first opening, the article further comprising:
a back bar extending from the first shoulder structure to a second shoulder structure, the second shoulder structure having a second opening provided therein;
wherein the article is adapted to facilitate a user
extending their left arm through the second opening; and
extending their right arm through the first opening.

14. The article of claim 13, wherein
the back bar is provided comprising an absorbent material or is provided with a detachable absorbent material.

15. The article of claim 1, further comprising a radial absorbent material detachably positioned toward the distal end of the compression sleeve, the radial absorbent material extending radially around at least a portion of the compression sleeve.

16. An article for providing sweat absorption for a user and providing compression to a body limb comprising:
a compression sleeve having a proximal end with an opening into which a user can insert a limb, and a distal end with an opening such that when the compression sleeve is fitted on the limb, the end of the limb protrudes from the distal end opening;
an absorbent material formed as a strip of material extending along a portion of the length of the compression sleeve, wherein a width of the absorbent material is less than a diameter of the compression sleeve and a length of the absorbent material is less than a length of the compression sleeve;
an anti-slip structure extending from the proximal end of the compression sleeve, the anti-slip structure comprising;
a portion of the proximal end of the compression sleeve extending longitudinally past the proximal opening and formed as a shoulder structure to form an upper end; and
an opening formed in the upper end;
wherein the article is adapted to facilitate a user extending their arm through the opening formed in the upper end and then extending their arm through the proximal opening of the compression sleeve such that the upper end is adapted to sit over top of the user's shoulder when the compression sleeve is fully advanced over the arm and the upper end over the shoulder, the upper end preventing the compression sleeve from moving downward relative to the user's arm when in use.

17. The article of claim 16, wherein the absorbent material is detachably connected to the compression sleeve.

18. The article of claim 17, further comprising a securing mechanism adapted to hold the absorbent material in a fixed position relative to the compression sleeve.

19. The article of claim 18, wherein the shoulder structure comprises a first shoulder structure and the opening in the upper end of the first shoulder structure comprises a first opening, the article further comprising:
a back bar extending from the first shoulder structure to a second shoulder structure, the second shoulder structure having a second opening provided therein;
wherein the article is adapted to facilitate a user extending their left arm through the second opening and extending their right arm through the first opening.

20. The article of claim 19, wherein the back bar is provided comprising an absorbent material or is provided with a detachable absorbent material.
